(19) United States
(12) Patent Application Publication
(10) Pub. No.: US 2018/0255797 A1
(43) Pub. Date: Sep. 13, 2018

(54) AROMA MILK COMPOSITION COMPRISING DIACETYL AND USES THEREOF

(71) Applicant: CHR. HANSEN A/S, Horsholm (DK)

(72) Inventors: Annamari JUKKOLA, Helsinki (FI); Saija LAAKSO, Salo (FI); Anja VALJUS-TIENSUU, Kerava (FI); Pirjo MERIMAA, Helsinki (FI); Kristiina KIVIHARJU, Vantaa (FI)

(21) Appl. No.: 15/950,659

(22) Filed: Apr. 11, 2018

Related U.S. Application Data
(62) Division of application No. 14/409,950, filed on Dec. 19, 2014.

(30) Foreign Application Priority Data
Jun. 29, 2012 (FI) 20125755

(51) Int. Cl. A23C 9/123; A23C 13/16; A23C 19/076; A23L 27/24; C12R 1/46; C12N 1/20; A23C 15/12; A23C 15/02

(52) U.S. Cl. CPC A23C 9/1234; A23Y 2220/49; A23C 13/16; A23C 19/0765; A23L 27/25; C12R 1/46; A23Y 2240/41; A23Y 2240/25; A23Y 2220/31; C12N 1/20; A23C 15/123; A23C 15/02

(57) ABSTRACT

The present invention relates to an aroma milk composition and a method of producing it. Further, the present invention relates to use of the aroma milk composition in the manufacture of a dairy product. Finally, the present invention relates to a new bacterial strain of Lactococcus lactis ssp. lactis.

This application is a divisional of Ser. No. 14/409,950 filed Dec. 19, 2014, which is the U.S. national phase of International Application No. PCT/FI2013/050715 filed Jun. 27, 2013 which designated the U.S. and claims priority to FI Patent Application No. 20125755 filed Jun. 29, 2012, the entire contents of each of which are hereby incorporated by reference.

### FIELD OF THE INVENTION

The present invention relates to an aroma milk composition and a method of producing it. In addition, the present invention relates to use of the aroma milk composition in the manufacture of a dairy product. Further, the present invention relates to a process for manufacturing butter. Finally, the present invention relates to a new bacterial strain of Lactococcus lactis ssp. lactis biovar. diacetylactis.

### BACKGROUND OF THE INVENTION

Butter and fat spread are foods in the form of an emulsion, mainly water-in-oil type emulsion, comprising principally an aqueous phase and edible fats and oils mainly composed of triglycerides of fatty acids of vegetable, animal, milk or marine origin. According to IDF standard fat spreads shall be classified into three groups i.e., milk fat products, mixed fat products and margarine products, based on the origin of the fat.

In milk fat products such as butter, the fat is 100% from milk fat. In mixed fat products, 10-80% of the total fat is milk fat and in margarine products only 3% (max.) of the total fat is milk fat. The maximum fat content shall be about 90%.

Butter is a dairy product, which has been made for centuries by churning and working fresh or fermented cream or milk. It is a water-in-oil emulsion, which contain at least 80% milk fat (also referring to as butterfat) and not more than 16% water, less than 2% of non-fat milk solids, and optionally salt. Butter is made as sweet cream butter or fermented cream butter, both of which can be salted. The aroma profiles of these two butters are very different. The aroma compounds of the fermented cream butter are formed by the fermentation of lactic acid bacteria, diacetyl being one of the most important ones.

Diacetyl is produced by the metabolism of a particular lactic acid bacterium, for instance, Lactococcus lactis ssp. lactis biov. diacetylactis-species. In the sugar metabolism by L. lactis-species glucose and lactose are converted to lactic acid more than 90%. Depending on the growth conditions L. lactis can also produce aroma compounds as diacetyl. The biosynthesis of these aroma compounds and lactic acid are closely related and they all have the same carbon source, pyruvate. Pyruvate can be metabolized from both glucose and citrate. LDH-enzyme can convert pyruvate to lactic acid. Without active LDH, acetate and α-acetolactate can be produced. α-acetolactate is a precursor for diacetyl, which is composed from that only by chemical oxidation. The metabolism route depends on the bacterial energy demand and demands for specific end products. The factors affecting diacetyl production are type of bacterial strain, oxygen, citrate, pH and temperature.

The aroma of soured butter is mainly composed of diacetyl, decalactone, butyric acid and capronic acid.

Traditionally, butter has been manufactured by separating milk into cream (about 40%) and heat-treating it. Then the cream is ripened and fermented using selected lactic acid bacteria and churned into butter. The butter manufacture has been simplified with different methods during decades. Fermenting/souring of butter can be made for example by adding selected lactic acid bacteria as such or together with naturally produced lactic acid directly to butter grains after churning. It is also known to use lot of diacetyl containing distillate produced by natural fermentation as such or with naturally produced lactic acid.

Butter is nowadays commonly produced by a process where sweet cream is turned into fermented butter by adding mixed starter(s) or starter distillate(s) after churning. The butter manufacturing process using starter distillate is described in FIG. 1.

Mixed starters are bacterial cultures containing several bacterial strains. They are commonly used in dairy industry. The mixed starters used in dairy products can be divided into mesophilic and thermophilic starters according to optimum growth temperature. Mesophilic starter cultures, composed of acid-forming lactococci and often of flavor producers, are used in the production of fermented cream butter, fermented milk products and of many cheese varieties. The starter cultures are usually composed of different species or of several strains of a single species

Major problem associated with the use of the conventional mesophilic cultures, such as lactococcal species, is bacteriophages infections which are common and has a large economic impact. Some strains that are strong producers of polysaccharides, for example exopolysaccharides, are sensitive to bacteriophages as well. Consequently, dramatic adverse effect on the texture of the final fermented product can then be seen.

Starter distillate (aroma distillate, lactic starter distillate) is a steam distillate of special lactic cultures, such as a culture of at least one of species Streptococcus lactis (now Lactococcus), S. cremoris, S. lactis subsp. diacetylactis, Leuconostoc citrovorum, and L. dextranicum grown on a medium consisting of skim milk and citric acid (0.1%). Starter distillate contains more than 98% water and a mixture of butterlike flavor compounds of which diacetyl is the major aroma compound (80 to 90%). Besides diacetyl, starter distillate typically contains acetaldehyde, ethyl formate, ethyl acetate, acetone, ethyl alcohol, 2-butanone, acetic acid, and acetoin. Depending on the diacetyl content and the intended use, the distillate is used in a dosage of 0.05 to 0.2%.

Bylund G, Butter and dairy spreads in Dairy processing handbook (Tetra Pak Processing Systems AB, Sweden, p 263-78, 1995) describes in detail different stages of processes for manufacturing butter and dairy spreads.

### BRIEF DESCRIPTION OF THE INVENTION

The present invention relates to a new aroma milk composition having a high amount of aroma compounds, especially diacetyl, which is an important aroma substance in dairy products such as butter. The aroma milk composition of the present invention comprises at least two diacetyl producing strains selected from Lactococcus lactis ssp. lactis biovar. diacetylactis strains and/or Lactococcus lactis strains, and diacetyl in an amount of at least 50 mg/kg, based on the weight of a milk based medium.

The aroma milk composition of the present invention is well suited for use as a starter culture and/or as an aroma forming agent, a flavouring agent, and/or an adjuvant.

The present invention also relates to a method of producing an aroma milk composition comprising at least one Lactococcus lactis ssp. lactis biovar. diacetylactis strain or L. lactis strain as diacetyl producing strain and diacetyl at least in an amount of 50 mg/kg in a milk based medium, based on the weight of the medium.

Further, the present invention relates to use of the aroma milk composition of the present invention in the manufacture of a dairy/food product, such as milk fat products, mixed fat products, margarine products, edible fat, fat mixture, butter, a spread, a fresh/soft cheese, a cottage cheese, a cream, a sour cream, a mayonnaise, sour milk, quark or another milk-based and/or milk-derived product or any other products desiring a natural buttery flavour.

Additionally, the present invention relates to a dairy/food or nutritional product comprising said aroma milk composition.

The present invention also relates to a method of manufacturing butter using an aroma milk composition comprising at least two diacetyl producing strains selected from Lactococcus lactis ssp. lactis biovar. diacetylactis strains and/or L. lactis strains, and diacetyl at least in an amount of 50 mg/kg.

The present invention further relates to Lactococcus lactis ssp. lactis biovar. diacetylactis strains and/or L. lactis strains producing diacetyl in an amount of 50 mg/kg at the minimum based on the weight of a milk based medium during 10-28 hours of cultivation.

In addition, the present invention relates to new bacterial strain Lactococcus lactis ssp. lactis biovar. diacetylactis DL2126, VTT E-123249 and to an aroma milk composition comprising Lactococcus lactis ssp. lactis biovar. diacetylactis DL2126, VTT E-123249.

The present invention further relates to an aroma milk composition comprising Lactococcus lactis ssp. lactis biovar. diacetylactis DL2126, VTT E-123249 as the diacetyl producing strain.

The present invention also relates to a method of manufacturing butter using Lactococcus lactis ssp. lactis biovar. diacetylactis DL2126, VTT E-123249 and/or an aroma milk composition comprising Lactococcus lactis ssp. lactis biovar. diacetylactis DL2126, VTT E-123249.

In addition, the present invention relates to a use of the strain Lactococcus lactis ssp. lactis biovar. diacetylactis DL2126, VTT E-123249 in food industry and to edible food or nutritional products, such as dairy products, which contain or which have been prepared by using said strain.

The objects of the invention are achieved by compositions, uses and methods characterized by what is stated in the independent claims. The preferred embodiments of the invention are disclosed in the dependent claims.

### BRIEF DESCRIPTION OF THE DRAWINGS

FIG. 1 shows a butter manufacturing process using starter distillate.

FIG. 2 shows the butter manufacturing process using the aroma milk composition of the present invention.

FIG. 3 shows the results of Example 1.

FIG. 4 shows the results of Example 2.

FIG. 5 shows the results of Example 3.

### DETAILED DESCRIPTION OF THE INVENTION

There is a continuous need for compositions which contain desired aroma compounds, such as diacetyl, and which can be used as a natural raw material and/or an ingredient in the manufacture of food products to produce natural butter flavor into the food products.

The invention is based on a finding that certain Lactococcus lactis ssp. lactis biovar. diacetylactis strains and Lactococcus lactis strains are able to produce aroma compounds, especially diacetyl, in high amounts into their milk based growth media. Accordingly, these Lactococcus lactis ssp. lactis biovar. diacetylactis cultures or L. lactis cultures (aroma milk compositions) can be used in the production of a food product by adding a required/desired amount of the culture to the product, optionally during the manufacturing process of said product or separately to otherwise finished product. These Lactococcus lactis ssp. lactis biovar. diacetylactis cultures or L. lactis cultures (aroma milk compositions) can be used as such or as in the form of a lysate, a concentrate, a granulate, a pellet or a lyophilisate.

The present invention relates to an aroma milk composition comprising at least two diacetyl producing strains selected from Lactococcus lactis ssp. lactis biovar. diacetylactis strains and/or L. lactis strains, and diacetyl in high amount i.e., in an amount of 50 mg/kg, at the minimum, in a milk based medium. In one embodiment, the aroma milk composition comprises at least one Lactococcus lactis ssp. lactis biovar. diacetylactis strain and at least one L. lactis strain, and diacetyl in an amount of 50 mg/kg, at the minimum, in a milk based medium. In another embodiment, the aroma milk composition comprises two diacetyl producing strains selected from Lactococcus lactis ssp. lactis biovar. diacetylactis strains and/or L. lactis strains and diacetyl in an amount of 50 mg/kg, at the minimum, in a milk based medium. In another embodiment, the aroma milk composition comprises one Lactococcus lactis ssp. lactis biovar. diacetylactis strain and one L. lactis strain and diacetyl in an amount of 50 mg/kg, at the minimum, in a milk based medium. In another embodiment, the aroma milk composition comprises two or more Lactococcus lactis ssp. lactis biovar. diacetylactis and/or L. lactis strains and diacetyl in an amount of 50 mg/kg, at the minimum, in a milk based medium. In another embodiment, an aroma milk composition comprises at least two diacetyl producing strains selected from Lactococcus lactis ssp. lactis biovar. diacetylactis strains and/or L. lactis strains, and diacetyl in an amount of 100 mg/kg, 150 mg/kg or 200 mg/kg, at the minimum, in the milk based medium. In another embodiment, an aroma milk composition comprises at least two Lactococcus lactis ssp. lactis biovar. diacetylactis strains or at least two L. lactis strains, and diacetyl in an amount of 50 mg/kg, 100 mg/kg, 150 mg/kg or 200 mg/kg, at the minimum, in the milk based medium. In an additional embodiment, the aroma milk composition comprises one Lactococcus lactis ssp. lactis biovar. diacetylactis strain and one L. lactis strain, and diacetyl in an amount of 100 mg/kg, 150 mg/kg or 200 mg/kg, at the minimum, in the milk based medium. In a certain embodiment, the aroma milk composition consists of one Lactococcus lactis ssp. lactis biovar. diacetylactis strain and one L. lactis strain, and contains diacetyl in an amount of 50 mg/kg, 100 mg/kg, 150 mg/kg or 200 mg/kg, at the minimum, in the milk based medium.

In one embodiment, the at least two diacetyl producing strains selected from Lactococcus lactis ssp. lactis biovar. diacetylactis strains and/or L. lactis strains are able to produce diacetyl in an amount of 50 mg/kg, at the minimum, during 10 to 28 hours cultivation in a milk based medium. In another embodiment, the at least two diacetyl producing strains selected from Lactococcus lactis ssp. lactis biovar. diacetylactis strains and/or L. lactis strains are able to produce diacetyl in an amount of 50 mg/kg, at the minimum, during 16 to 28 hours cultivation in a milk based medium. In a further embodiment, the at least two diacetyl producing strains selected from Lactococcus lactis ssp. lactis biovar. diacetylactis strains and/or L. lactis strains are able to produce diacetyl in an amount of 50 mg/kg, at the minimum, during 20 to 26 hours cultivation in a milk based medium. In a certain embodiment, the aroma composition comprises Lactococcus lactis ssp. lactis biovar. diacetylactis DL2126, VTT E-123249.

In the aroma milk composition of the present invention Lactococcus lactis ssp. lactis biovar. diacetylactis and/or L. lactis strain(s) can be either viable and active or living or non-viable (inactive) or dead (heat-killed, sonicated, shocked, irradiated or lysed). The sensory characteristics of butter manufactured with aroma milk composition containing an inactivated Lactococcus lactis ssp. lactis biovar. diacetylactis strain did not differ essentially from butter manufactured with aroma milk composition containing an active Lactococcus lactis ssp. lactis biovar. diacetylactis strain.

The aroma milk composition of the invention may also include other microorganisms contained in starters used in the food industry, specifically in the dairy industry. There are numeral well-documented strains of starters, which are commercially available from producers such as Chr Hansen A/S, Denmark, and Danisco/Wiesby GmbH, Germany.

The present invention further relates to Lactococcus lactis ssp. lactis biovar. diacetylactis strains or L. lactis strains producing diacetyl in an amount of 50 mg/kg, at the minimum, based on the weight of a milk based medium during 10 to 28 hours of cultivation. In one embodiment, a Lactococcus lactis ssp. lactis biovar. diacetylactis strain or a L. lactis strain produces diacetyl in an amount of 100 mg/kg, 150 mg/kg or 200 mg/kg, at the minimum, based on the weight of a milk based medium during 10 to 28 hours of cultivation. In another embodiment, a Lactococcus lactis ssp. lactis biovar. diacetylactis strain or a L. lactis strain produces diacetyl in an amount of 50 mg/kg at the minimum based on the weight of a milk based medium during 16 to 28 hours of cultivation. In another embodiment, a Lactococcus lactis ssp. lactis biovar. diacetylactis strain or a L. lactis strain produces diacetyl in an amount of 100 mg/kg, 150 mg/kg or 200 mg/kg, at the minimum, based on the weight of a milk based medium during 16 to 28 hours cultivation in a milk based medium. In a further embodiment, a Lactococcus lactis ssp. lactis biovar. diacetylactis strain or a L. lactis strain produces diacetyl in an amount of 50 mg/kg at the minimum based on the weight of a milk based medium during 20 to 26 hours of cultivation. In another further embodiment, a Lactococcus lactis ssp. lactis biovar. diacetylactis strain or a L. lactis strain produces diacetyl in an amount of 100 mg/kg, 150 mg/kg or 200 mg/kg, at the minimum, based on the weight of a milk based medium during 20 to 26 hours of cultivation.

The present invention further relates to strains Lactococcus lactis ssp. lactis biovar. diacetylactis and L. lactis producing together diacetyl in an amount of 50 mg/kg at the minimum based on the weight of a milk based medium during 10 to 28 hours of cultivation. In one embodiment, a Lactococcus lactis ssp. lactis biovar. diacetylactis strain and a L. lactis strain produce diacetyl in an amount of 100 mg/kg, 150 mg/kg or 200 mg/kg, at the minimum, based on the weight of a milk based medium during 10 to 28 hours of cultivation. In one embodiment, a Lactococcus lactis ssp. lactis biovar. diacetylactis strain and a L. lactis strain produce diacetyl in an amount of 50 mg/kg, at the minimum, based on the weight of a milk based medium during 16 to 28 hours of cultivation. In another embodiment, a Lactococcus lactis ssp. lactis biovar. diacetylactis strain and a L. lactis strain produce diacetyl in an amount of 100 mg/kg, 150 mg/kg or 200 mg/kg, at the minimum, based on the weight of a milk based medium during 16 to 28 hours of cultivation. In a further embodiment, a Lactococcus lactis ssp. lactis biovar. diacetylactis strain and a L. lactis strain produce diacetyl in an amount of 50 mg/kg at the minimum based on the weight of a milk based medium during 20 to 26 hours of cultivation. In another further embodiment, a Lactococcus lactis ssp. lactis biovar. diacetylactis strain and a L. lactis strain produce diacetyl in an amount of 100 mg/kg, 150 mg/kg or 200 mg/kg, at the minimum, based on the weight of a milk based medium during 20 to 26 hours of cultivation.

In a further embodiment, the Lactococcus lactis ssp. lactis biovar. diacetylactis strain or the L. lactis strain is not a metabolically modified mutant. In another further embodiment, the Lactococcus lactis ssp. lactis biovar. diacetylactis strain or L. lactis strain is a metabolically modified mutant. Metabolic modification includes NAHD-oxidase (NOX) overproduction and/or α-acetolactate decarboxylase (ALDB) inactivation, for example.

As used herein, the term “milk based medium” (or milk based growth medium) refers to a medium containing milk, whey or combinations of milk and whey as such obtained from a milk producing animal (a cow, a sheep, a goat, a camel or a mare, for example) or pre-treated/pre-processed as desired, for example as a concentrate or as a hydrolysate or as enzyme treated milk or whey. The milk based medium can be prepared from one or more components obtained from milk and/or whey by various separation techniques, membrane techniques or combinations thereof, such as micro- and ultrafiltration permeates. The milk based medium may also contain whey or casein hydrolysate(s). The milk based medium for culturing the Lactococcus lactis ssp. lactis biovar. diacetylactis and/or L. lactis strain(s) and producing the aroma milk composition comprises in addition to lactose typically also citrate, yeast extract and/or casein hydrolysate.

The present invention is also directed to a method of producing an aroma milk composition comprising at least one diacetyl producing strain selected from Lactococcus lactis ssp. lactis biovar. diacetylactis strains and/or L. lactis strains, and diacetyl in an amount of at least 50 mg/kg in a milk based medium, which method comprises

providing at least one Lactococcus lactis ssp. lactis biovar. diacetylactis strain or L. lactis strain and a growth medium comprising lactose, citrate, yeast extract and/or casein hydrolysate, and mixing said strain with said medium; and

incubating the mixture thus obtained for 10 to 28 hours in conditions optimized with regard to aeration and temperature to provide the aroma milk composition comprising at least one Lactococcus lactis ssp. lactis biovar. diacetylactis strain and/or L. lactis strain, and diacetyl in an amount of 50 mg/kg at the minimum based on the weight of a milk based medium.

In a certain embodiment, the invention is directed to a method of producing an aroma milk composition comprising at least one diacetyl producing strain selected from Lactococcus lactis ssp. lactis biovar. diacetylactis strains and/or L. lactis strains, and diacetyl in an amount of at least 100 mg/kg, 150 mg/kg or 200 mg/kg in a milk based medium.

In a certain embodiment, the method of producing an aroma milk composition comprises one diacetyl producing strain selected from Lactococcus lactis ssp. lactis biovar. diacetylactis strains or L. lactis strains. In a certain embodiment, the method of producing an aroma milk composition comprises at least two diacetyl producing strains selected from Lactococcus lactis ssp. lactis biovar. diacetylactis strains and/or L. lactis strains. In a certain embodiment, the method of producing an aroma milk composition comprises two diacetyl producing strains selected from Lactococcus lactis ssp. lactis biovar. diacetylactis strains or L. lactis strains. In one embodiment, the method of producing an aroma milk composition comprises Lactococcus lactis ssp. lactis biovar. diacetylactis strain(s). In another embodiment, the method of producing an aroma milk composition comprises one Lactococcus lactis ssp. lactis biovar. diacetylactis strain and one L. lactis strain. In a certain embodiment, the method of producing an aroma milk composition comprises Lactococcus lactis ssp. lactis biovar. diacetylactis DL2126, VTT E-123249.

In one embodiment, the incubation/culturing time is in the range of 16 to 28 hours. In another embodiment the incubation/culturing time is in the range of 20 to 26 hours.

In one embodiment, the citrate concentration of the growth medium is in the range of 0.80-1.40%. In another embodiment, the citrate concentration of the growth medium is in the range of 1.00-1.10%.

In one embodiment, the yeast extract and/or casein hydrolysate concentration of the growth medium is in the range of 0-0.5%. In another embodiment, the yeast extract and/or casein hydrolysate concentration of the growth medium is in the range of 0.25-0.5%.

In one embodiment, the aeration is in the range of 0.3-1.1 vvm (vvm=gas volume flow per unit of liquid volume per minute (volume per volume per minute)). In another embodiment, the aeration is in the range of 0.4-0.9 vvm. In another embodiment, the aeration is in the range of 0.55-0.9 vvm. In the aeration, the culture is mixed to dissolve the oxygen using mixers, mixing rates and other mixing conditions known by a person skilled in the art.

In one embodiment, the temperature is in the range of 27 to 33° C. In another embodiment temperature is in the range of 29 to 31° C.

In a further embodiment, the method of producing an aroma milk composition comprising at least one Lactococcus lactis ssp. lactis biovar. diacetylactis strain and/or L. lactis strain and diacetyl in an amount of 50 mg/kg, at the minimum, based on the weight of a milk based medium comprises

providing at least one Lactococcus lactis ssp. lactis biovar. diacetylactis strain and/or L. lactis strain and a growth medium comprising lactose, citrate, yeast extract and/or casein hydrolysate, and mixing said strain with said medium; and

incubating the mixture thus obtained for 16-28 hours wherein the citrate concentration is 1.00-1.10%, the concentration of yeast extract and/or casein hydrolysate is in the range of 0.25-0.5%, the aeration is in the range of 0.4-0.9 vvm, and the temperature is in the range of 29 to 31° C. to provide said aroma milk composition.

In one embodiment, the pH of the aroma milk composition is in the range of 4.8-5.4. In another embodiment, the pH of the aroma milk composition is in the range of 5.0-5.2.

The ability of a Lactococcus lactis ssp. lactis biovar. diacetylactis strain or a L. lactis strain to produce high amount of diacetyl is important and useful for example in the manufacture of dairy products, such as butter, a spread, a fresh/soft cheese, a cream, a mayonnaise or sour milk. Thus, the aroma milk composition of the present invention can be used in the manufacture of food/dairy products for providing taste (flavor) of butter. Depending on the diacetyl content of the aroma milk composition, the intended use and the dairy product, the aroma milk composition is typically used in a dosage of 0.3 to 3%.

Specifically, the aroma milk composition of the present invention can be used in the manufacture of butter instead of a starter distillate and/or a mixed starter. Accordingly, the present invention relates to a use of the aroma milk composition in the manufacture of butter.

Further, the present invention relates to a process for manufacturing butter. Butter is made as sweet cream butter or fermented (or soured or cultured or lactic) cream butter, both of which can be salted.

In one embodiment, a process for manufacturing butter from raw material milk is as follows:

a) raw material milk is separated into cream containing about 40% fat and to a non-fat milk,

b) the non-fat milk obtained in step (a) is optionally used to produce an aroma milk composition,

c) the cream obtained in step (a) is pasteurized and optionally ripened,

d) then the cream is churned to separate butter grains and buttermilk,

e) the butter grains obtained in the step (d) are worked,

f) aroma milk composition, optionally produced from the non-fat milk obtained from step (a), is added to the grains, standardized, and worked to butter, and

g) optionally the butter product is packed.

In another embodiment, a process for manufacturing butter from raw material milk is as follows:

a) raw material milk is separated into cream containing about 40% fat and to a non-fat milk,

b) the non-fat milk obtained in step (a) is used to produce an aroma milk composition,

c) the cream obtained in step (a) is pasteurized and optionally ripened,

d) then the cream is churned to separate butter grains and buttermilk,

e) the butter grains obtained in the step (d) are worked,

f) aroma milk composition produced from the non-fat milk obtained from step (a), is added to the grains, standardized, and worked to butter, and

g) optionally the butter product is packed.

Separation, pasteurization, ripening, churning, working and standardization are done using typical methods and conditions known by the person skilled in the art.

In one embodiment, the aroma milk composition comprises at least two diacetyl producing strains selected from Lactococcus lactis ssp. lactis biovar. diacetylactis strains and/or L. lactis strains, and diacetyl in an amount of at least 50 mg/kg in a milk based medium. In another embodiment, the aroma milk composition comprises at least two diacetyl producing strains selected from Lactococcus lactis ssp. lactis biovar. diacetylactis strains and/or L. lactis strains, and diacetyl in an amount of at least 100 mg/kg, 150 mg/kg or 200 mg/kg in a milk based medium. In a certain embodiment, the aroma milk composition comprises two diacetyl producing strains selected from Lactococcus lactis ssp. lactis biovar. diacetylactis strains or L. lactis strains and diacetyl in an amount of 50 mg/kg, 100 mg/kg, 150 mg/kg or 200 mg/kg, at the minimum, in a milk based medium. In another embodiment, the aroma milk composition comprises one Lactococcus lactis ssp. lactis biovar. diacetylactis strain and one L. lactis strain and diacetyl in an amount of 50 mg/kg, 100 mg/kg, 150 mg/kg or 200 mg/kg, at the minimum, in a milk based medium. In a further embodiment, the aroma milk composition comprises at least one diacetyl producing strain selected from Lactococcus lactis ssp. lactis biovar. diacetylactis strains or L. lactis strains and diacetyl in an amount of 50 mg/kg, 100 mg/kg, 150 mg/kg or 200 mg/kg, at the minimum, in a milk based medium. In one embodiment, the aroma milk composition comprises Lactococcus lactis ssp. lactis biovar. diacetylactis strain(s). In a certain embodiment, the aroma milk composition comprises Lactococcus lactis ssp. lactis biovar. diacetylactis DL2126, VTT E-123249.

The aroma milk composition can be used together with or instead of a starter distillate in a butter manufacture process. In addition, the aroma milk composition can be used together with or instead of a mixed starter in a butter manufacture process. In one embodiment, the aroma milk composition is used instead of a starter distillate in a butter manufacture process. In another embodiment, the aroma milk composition is used instead of a mixed starter in a butter manufacture process.

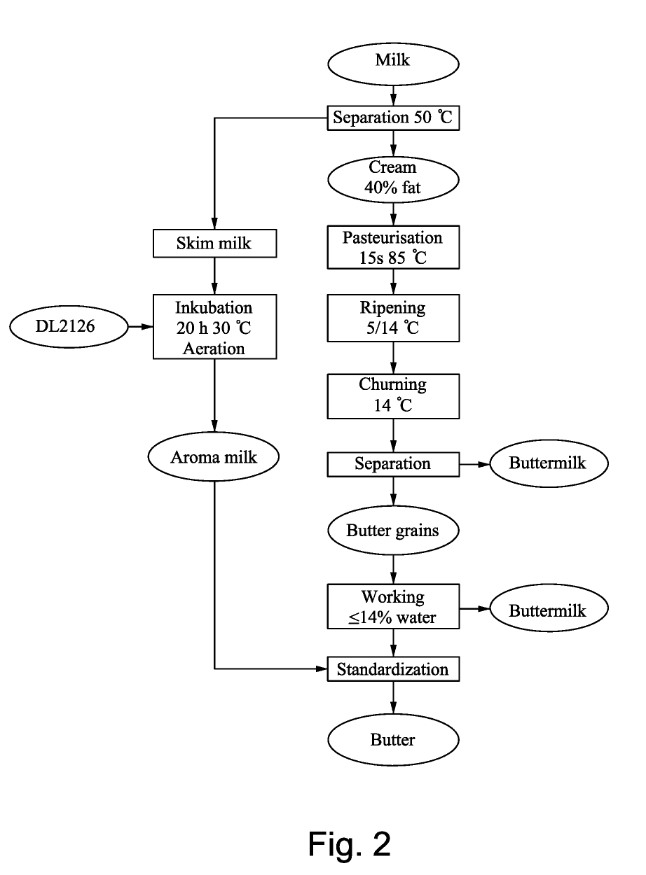

A butter manufacturing process with the aroma milk composition of the present invention is described in FIG. 2.

Correspondingly, in the manufacture of other dairy products, such as milk fat products, mixed fat products, margarine products, edible fats, fat mixtures, spreads, fresh/soft cheeses, cottage cheeses, creams, sour creams, mayonnaises and sour milks, techniques known for these dairy products per se are generally used as far as various process conditions, such as temperature, time, mixing, and heat treatment, are concerned.

In the manufacture of spreads, the manufacture technique is a known emulsification technology combined with surface scraper crystallization, i.e.

margarine technology. Said technology is commonly used in the manufacture of margarines, fat mixtures and recombined products within a wide range of fat content (e.g. 25 to 90%).

The manufacture of spreads may be divided into the following parts: preparation of an aqueous phase and a fat phase, emulsification, pasteurization, cooling/crystallization, and packaging. The ingredients are weighed and mixed as a batch, processed and cooled continuously. The smaller ingredients, such as emulsifiers, stabilizers, salt, preservatives, colour, aroma and vitamins, are dispersed in to the phases according to solubility. The aqueous phase contains the water-soluble ingredients while the fat phase contains the fat-soluble ingredients. In terms of manufacture technology, it is characteristic of the manufacture of a spread that the ingredients are added in a certain order.

In these processes the aroma milk composition can be added to the process in an appropriate process step or stage known by a person skilled in the art.

Fat mixtures and spreads are, as is well known, manufactured using either a mixer technique or an emulsion technique.

The aroma milk composition can be added to the butter component used as a raw material in the manufacture of fat mixture/blends and blended spreads. Alternatively, it can be added during the mixing phase to the emulsion or to the product flow.

The present invention is further directed to a dairy product, which comprises an aroma milk composition comprising at least two diacetyl producing strains selected from Lactococcus lactis ssp. lactis biovar. diacetylactis strains and/or L. lactis strains, and diacetyl in an amount of 50 mg/kg, 100 mg/kg, 150 mg/kg or 200 mg/kg, at the minimum, based on the weight of a milk based medium. In one embodiment, the dairy product comprises an aroma milk composition comprising at least one Lactococcus lactis ssp. lactis biovar. diacetylactis strain and at least one L. lactis strain, and diacetyl in high amount i.e., in an amount of 50 mg/kg, 100 mg/kg, 150 mg/kg or 200 mg/kg, at the minimum, in a milk based medium. In another embodiment, the dairy product comprises an aroma milk composition comprising two diacetyl producing strains selected from Lactococcus lactis ssp. lactis biovar. diacetylactis strains and/or L. lactis strains and diacetyl in an amount of 50 mg/kg, 100 mg/kg, 150 mg/kg or 200 mg/kg, at the minimum, in a milk based medium. In another embodiment, the dairy product comprises an aroma milk composition comprising one Lactococcus lactis ssp. lactis biovar. diacetylactis strain and one L. lactis strain, and diacetyl in an amount of 50 mg/kg, 100 mg/kg, 150 mg/kg or 200 mg/kg, at the minimum, in a milk based medium. In another embodiment, the dairy product comprises an aroma milk composition comprising two or more Lactococcus lactis ssp. lactis biovar. diacetylactis and/or L. lactis strains, and diacetyl in an amount of 50 mg/kg, 100 mg/kg, 150 mg/kg or 200 mg/kg, at the minimum, in a milk based medium. In a further embodiment, the dairy product comprises an aroma milk composition comprising at least two Lactococcus lactis ssp. lactis biovar. diacetylactis strains or at least two L. lactis strains, and diacetyl in an amount of 50 mg/kg, 100 mg/kg, 150 mg/kg or 200 mg/kg, at the minimum, in the milk based medium. In a certain embodiment, the dairy product comprises an aroma milk composition comprising Lactococcus lactis ssp. lactis biovar. diacetylactis DL2126, VTT E-123249.

The present invention is also directed to a dairy product produced by using said aroma milk composition. In one embodiment of the present invention, the dairy product is a milk fat product, a mixed fat product, a margarine product, an edible fat, a fat mixture, butter, a spread, a fresh/soft cheese, a cream, a mayonnaise or sour milk. In another embodiment, the dairy product is butter. The products of the present invention have butter like taste and/or aroma (natural butter flavour).

The dairy products, such as butter, cream, cooking cream, sour cream, fresh cheese and mixed fat spread, comprising an aroma milk composition comprising at least two diacetyl producing strains selected from Lactococcus lactis ssp. lactis biovar. diacetylactis strains and/or L. lactis strains, and diacetyl in an amount of 50 mg/kg at the minimum based on the weight of a milk based medium, were found to have palatable sensory characteristics. With the aroma milk composition of the present invention it is possible to include in dairy products, such as cream, cooking cream, sour cream, fresh cheese, mixed fat spread, butter like flavor and aroma and/or balanced taste. Further, it was found that the aroma milk composition of the present invention did not cause any adverse tastes to the products or interfere with the conventional manufacturing processes.

In addition, the present invention is directed to the strain Lactococcus lactis ssp. lactis biovar. diacetylactis DL2126, which is able to produce aroma compounds, especially diacetyl in an amount of 50 mg/kg, at the minimum, in its growth medium during 10 to 28 hours of cultivation. Lactococcus lactis ssp. lactis biovar. diacetylactis DL2126 has been deposited on Jun. 20, 2012, with the depository authority VTT Culture Collection, P.O. Box 1000 (Tietotie 2), FI-02044 VTT, Espoo, Finland (VTTCC) under accession number VTT E-123249.

Lactococcus lactis ssp. lactis biovar. diacetylactis DL2126 (VTT E-123249) is Gram-positive, facultative anaerobe, cocci in short chains, mesophilic (with optimum temperature of about 30° C.). The strain ferments at least the following carbohydrates (sugars, sugar alcohols): D-ribose, D-galactose, D-glucose, D-fructose, D-mannose, N-acetylglucosamine, arbutin, esculin, salicin, D-cellobiose, D-maltose, D-lactose, D-trehalose, amidon and gentiobiose (API 50 CHL, bioMerieux).

Lactococcus lactis ssp. lactis biovar. diacetylactis DL2126 (VTT E-123249) can be cultured in MRS-broth (deMan, J. C., M. Rogosa, and M. E. Sharpe. J. Bacteriol. 23:130. 1960), in Nickels and Leesment medium (ISO 17792/IDF 180 Standard, Milk, milk products and mesophilic starter cultures—Enumeration of citrate-fermenting lactic acid bacterial—Colony-count technique at 25° C.) or in modified KCA broth (Saxelin Maija-Liisa, et al., 1986. Applied and Environmental Microbiology 52:771-777).

In the present invention it was surprisingly found that Lactococcus lactis ssp. lactis biovar. diacetylactis DL2126, VTT E-123249 was able to produce diacetyl in an amount of at least four times higher than the starters known in the art in a medium containing lactose and citrate when each strain/starter culture was cultured in its optimal conditions. Lactococcus lactis ssp. lactis biovar. diacetylactis DL2126, VTT E-123249 was found to be much stronger producer of diacetyl than commercially available culture Probat 505 FRO 500 DCU (Danisco A/S, Denmark), a mixed starter containing Lactococcus lactis subsp. lactis, Lactococcus lactis subsp. cremoris, Lactococcus lactis subsp. lactis biovar. diacetylactis and Leuconostoc mesenteroides subsp. cremoris. Furthermore, Lactococcus lactis ssp. lactis biovar. diacetylactis DL2126 (VTT E-123249) was found to be more potential producer of diacetyl than commercially available mixed starter C27 (CSK, The Netherlands).

In regulated conditions (citrate concentration 1.0-1.1%, aeration 0.4-0.9 vvm, 29-31° C.) Lactococcus lactis ssp. lactis biovar. diacetylactis DL2126 VTT E-123249 produced diacetyl in an amount of 100 mg/kg, 150 mg/kg, 200 mg/kg and even in an amount of 240 mg/kg during a cultivation of 20 to 26 hours. Determination of diacetyl was made with static HS-GC-MS (headspace-gas chromatography-mass spectrometry) gas chromatography described in Miettinen S M et al., 2002, J Agric. Food Chem. 50, 4232-9.

The present invention is also directed to an aroma milk composition comprising Lactococcus lactis ssp. lactis biovar. diacetylactis DL2126, VTT E-123249. In one embodiment, the aroma milk composition comprises only Lactococcus lactis ssp. lactis biovar. diacetylactis DL2126, VTT E-123249 as the diacetyl producing strain. In another embodiment, the aroma milk composition comprises in addition to Lactococcus lactis ssp. lactis biovar. diacetylactis DL2126, VTT E-123249 at least one other Lactococcus lactis ssp. lactis biovar. diacetylactis strain. In a further embodiment, the other Lactococcus lactis ssp. lactis biovar. diacetylactis strain is a strain able to produce diacetyl in an amount of 50 mg/kg at the minimum during 10 to 28 hours of cultivation. In another embodiment, the aroma milk composition comprises in addition to Lactococcus lactis ssp. lactis biovar. diacetylactis DL2126, VTT E-123249 at least one Lactococcus lactis strain. In another embodiment, the aroma milk composition comprises in addition to Lactococcus lactis ssp. lactis biovar. diacetylactis DL2126 (VTT E-123249), one Lactococcus lactis strain. In a further embodiment, the L. lactis strain is a strain able to produce diacetyl in an amount of 50 mg/kg, at the minimum, during 10 to 28 hours of cultivation. In a certain embodiment, the aroma milk composition comprises strains Lactococcus lactis ssp. lactis biovar. diacetylactis DL2126 (VTT E-123249), and Lactococcus lactis 1002 (Valio Oy). The aroma milk composition of the invention may also include other microorganisms contained in starters used in the food industry, specifically in the dairy industry.

In the aroma milk composition of the present invention, Lactococcus lactis ssp. lactis biovar. diacetylactis DL2126 VTT E-123249 can be either viable and active or living or non-viable (inactive) or dead (heat-killed, sonicated, shocked, irradiated or lysed).

The aroma milk composition comprising Lactococcus lactis ssp. lactis biovar. diacetylactis DL2126 VTT E-123249 can be used in the manufacture of food/dairy products for providing taste (flavor) of butter. In one embodiment, the present invention relates to a use of Lactococcus lactis ssp. lactis biovar. diacetylactis DL2126 VTT E-123249 and/or the aroma milk composition comprising Lactococcus lactis ssp. lactis biovar. diacetylactis DL2126 VTT E-123249 in the manufacture of butter. Further, the present invention relates to a method for manufacturing butter using Lactococcus lactis ssp. lactis biovar. diacetylactis DL2126 VTT E-123249 and/or the aroma milk composition comprising Lactococcus lactis ssp. lactis biovar. diacetylactis DL2126 VTT E-123249. In one embodiment of the invention, Lactococcus lactis ssp. lactis biovar. diacetylactis DL2126 VTT E-123249 and/or the aroma milk composition comprising Lactococcus lactis ssp. lactis biovar. diacetylactis DL2126 VTT E-123249 is used instead of a starter distillate or a mixed starter in a butter manufacturing process. In another embodiment, Lactococcus lactis ssp. lactis biovar. diacetylactis DL2126 VTT E-123249 and/or the aroma milk composition comprising Lactococcus lactis ssp. lactis biovar. diacetylactis DL2126 VTT E-123249 is used together with a starter distillate or mixed starter in a butter manufacturing process.

The present invention is further directed to a dairy product comprising Lactococcus lactis ssp. lactis biovar. diacetylactis DL2126 VTT E-123249 and/or the aroma milk composition comprising Lactococcus lactis ssp. lactis biovar. diacetylactis DL2126, VTT E-123249. The present invention is also directed to a dairy product produced by using Lactococcus lactis ssp. lactis biovar. diacetylactis DL2126 VTT E-123249 and/or the aroma milk composition comprising Lactococcus lactis ssp. lactis biovar. diacetylactis DL2126, VTT E-123249. In one embodiment of the present invention, the dairy product is a milk fat product, a mixed fat product, a margarine product, an edible fat, a fat mixture, butter, a spread, a fresh/soft cheese, a cream, a mayonnaise or sour milk. In another embodiment, the dairy product is butter.

The term “dairy product” is intended to cover all consumable milk-based products that can be solid, jellied or liquid which are produced by using the aroma milk composition of the invention as an aroma forming compound, as a starter alone or in combination with conventional starters. The dairy product can be derived from, e.g., cow's milk, goat's milk, sheep's milk, skimmed milk, whole milk or milk recombined from powdered milk.

In the preparation of the dairy products, the optimum conditions for an economic, inexpensive and efficient production process are employed. For example, conventional heat treatment methods such as pasteurization (heating for example at about 72° C. for at least 15 seconds), ESL treatment (heating for example at about 130° C. for 1 to 2 seconds), UHT treatment (heating for example at about 138° C. for 2 to 4 seconds) or high temperature pasteurization (heating at 95° C. for 5 minutes), are employed.

In one embodiment of the invention, the aroma milk composition of the invention is used in the preparation of a fermented dairy product.

The fermentation conditions such as, starter culture(s), temperature, pH and time for the production of fermented milk products or ingredients are selected to meet the requirements of the final product. The selection of suitable conditions belongs to knowledge of a person skilled in the art.

The dairy product could be produced by using the conventional fermentation procedures of the dairy industry or alternatively, the product can be soured with a chemical acidifying agent.

The following examples illustrate the present invention. The examples are not to be construed to limit the claims in any manner whatsoever.

### EXAMPLE 1

### Production of Diacetyl

Strains Lactococcus lactis ssp. lactis biovar. diacetylactis DL2126 (VTT E-123249), and Lactococcus lactis 1002 (Valio Oy) were fermented in 500 ml recomposed milk medium supplemented with 0.25% casein hydrolysate in the temperature of 30° C. and with 400 rpm mixing. Further 0.2 ml structol was added for preventing foaming.

In the fermentation of the strain DL 2126 citrate concentration was 1.1% and the aeration was 275 ml/min whereas for the strain 1002 citrate concentration was 1.3% and the aeration was 295 ml/min.

Commercially available starters Probat 505 FRO 500 DCU (Danisco) ja C27 (CSK) were fermented in 500 ml recomposed milk medium supplemented with 0.25% casein hydrolysate in the temperature of 30° C. and with 50 or 150 rpm mixing depending on the aeration. The aeration was 0-50 ml/min. Further 0.2 ml structol was added for preventing foaming. In the fermentation of Probat 505 FRO 500 DCU 6 citrate concentration was 1.1% and C27-stater citrate concentration was 1.4%.

Strains Lactococcus lactis ssp. lactis biovar. diacetylactis DL2126 (VTT E-123249) and L. lactis 1002 produced diacetyl in amounts of 200 mg/kg and 60 mg/kg, respectively whereas the amounts produced by the commercial starters Probat 505 FRO 500 DCU and C27 were 20 mg/kg and 29 mg/kg, respectively.

Determination of diacetyl was made with static HS-GC-MS (headspace-gas chromatography-mass spectrometry) gas chromatography as described in Miettinen S. M., et al., 2002, J Agric. Food Chem. 50, 4232-9.

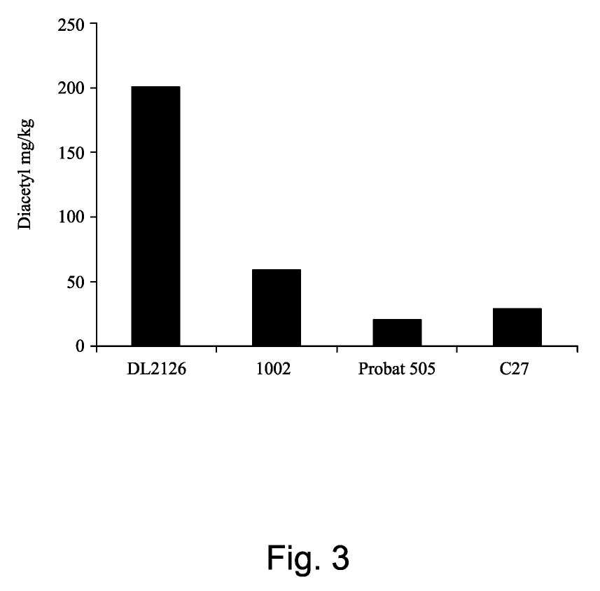

The results are presented in FIG. 3.

### EXAMPLE 2

### Sensory Characteristics of the Butters

The butters were manufactured according to the process described in FIG. 2, wherein either 1% or 2% of the aroma milk composition comprising Lactococcus lactis ssp. lactis biovar. diacetylactis DL2126 VTT E-123249 was added to the butter grains after working.

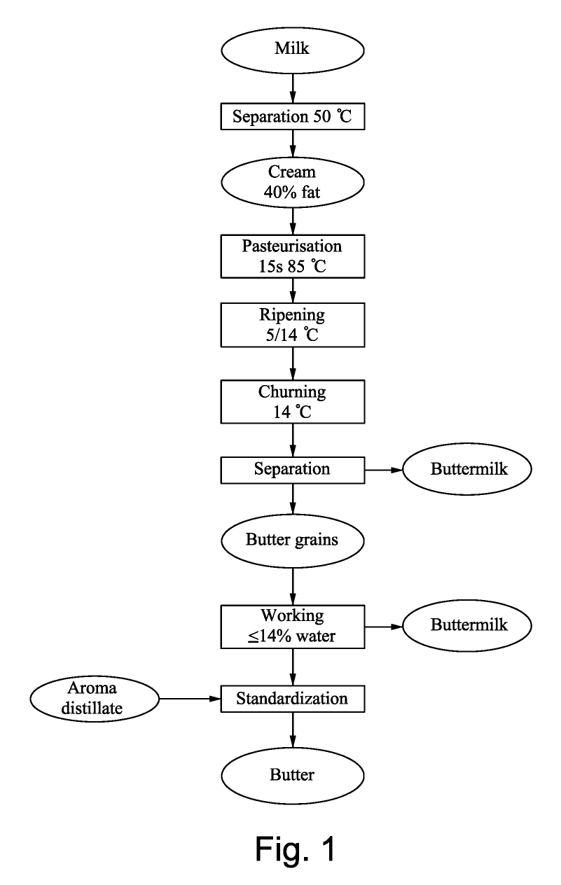

The buttery taste, freshness, acidity and savor of these two butters were compared with a reference butter produced according to the process described in FIG. 1 using a commercial starter distillate. The value for the buttery taste, freshness, acidity and savor of this reference product was set to 0.

The evaluation was done by a panel of 6 panelists trained to taste and evaluate butters and fat spreads.

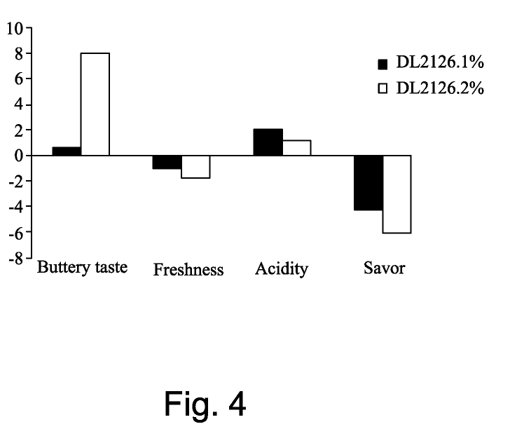

The results are presented in FIG. 4.

### EXAMPLE 3

### Diacetyl Contents of Butters

Diacetyl amounts in butters containing aroma milk composition comprising Lactococcus lactis ssp. lactis biovar. diacetylactis DL2126 (VTT E-123249) were measured with static HS-GC-MS (headspace-gas chromatography-mass spectrometry) gas chromatography as described in Miettinen et al., 2002, J Agric. Food Chem. 50, 4232-9.

DL2126 butter (churned) was made in traditional batch churn wherein the aroma milk composition was added to the butter grains in the churn. DL2126 butter (mixed) was made by mixing the aroma milk composition with finished butter, which did not contain starter distillate. Reference sample was a commercially available butter with was manufactured with starter distillate.

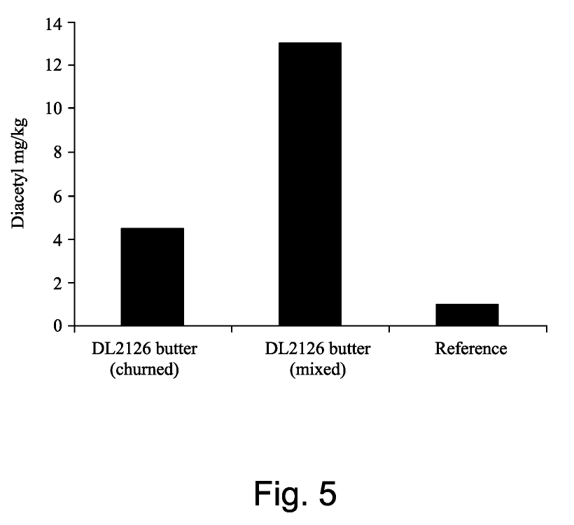

DL2126 butter (churned) contained diacetyl in an amount of about 5 mg/kg. DL2126 butter (mixed) contained diacetyl in an amount of 13 mg/kg whereas the reference butter contained diacetyl in an amount of less than 1 mg/kg. The results are presented in FIG. 5.

### EXAMPLE 4

### Sensory Characteristics of Some Dairy Products

The aroma milk composition of the present invention was added to commercially available cream (double cream, 35% fat), cooking cream (15% fat), sour cream (10% fat, fresh cheese (13% fat), quark (0.3% fat), fat spread (82% fat). After the addition, the products were stored in the temperature of 5° C.

TABLE 1

| Product | The addition of the aroma milk composition | | | |
|---|---|---|---|---|
| cream (double cream, 35%) | 0.5% | 1.0% | 1.5% | |
| cooking cream (15%) | 0.5% | 1.0% | 1.5% | |
| sour cream (10%) | 0.5% | 1.0% | 1.5% | |
| fresh cheese (13%) | 0.5% | 1.0% | 1.5% | |
| quark (0.3%) | 0.5% | 1.0% | 1.5% | |
| fat spread (82% 80/20) | 0.7% | 1.3% | 2.0% | 2.5% |

The sensory evaluations were made after five days of storage by comparing the aroma milk composition containing products with similar products without any addition of the aroma milk composition.

These evaluations showed that in neutral products, such as cream, cooking cream and fat spread, the aroma milk composition provided flavored and butter like taste to the products. Also clear differences in the flavors of these products were observed which also increased with the increasing amount of the aroma milk composition. There were no big differences between the different additions of the aroma milk composition with regard to the taste of the products. The aroma milk composition did not coagulate the creams.

In soured products such as fresh cheese, quark and sour cream, the aroma milk composition clearly rounded the taste, balanced and made the products full flavored. In each product group the sample produced with the minimum amount of the aroma milk composition differed clearly from the reference product which was not supplemented with the aroma milk composition. Big differences between the added amounts of the aroma milk composition were not observed.

### EXAMPLE 5

### Production of the Aroma Milk Composition

The aroma milk compositions comprising the strain Lactococcus lactis ssp. lactis biovar. diacetylactis DL2126 VTT E-123249 were produced by culturing the strain in a milk based growth medium regulated with regard to the citrate concentration (1.0-1.1%), aeration (0.4-0.9 vvm) and temperature (29-31° C.) for 20 to 26 hours.

Diacetyl contents of the milk aroma compositions were 100 mg/kg, 150 mg/kg, 200 mg/kg and 240 mg/kg depending on the exact conditions.

Determination of diacetyl was made with static HS-GC-MS (headspace-gas chromatography-mass spectrometry) gas chromatography described in Miettinen S. M. et al., 2002, J Agric. Food Chem. 50, 4232-9.

### EXAMPLE 6

The Aroma Milk Composition with Selected Diacetyl Producing Strain(s)

The aroma milk compositions comprising the strain Lactococcus lactis ssp. lactis biovar. diacetylactis DL2126 VTT E-123249 (composition A), the strain Lactococcus lactis ssp. lactis biovar. diacetylactis DL2126 VTT E-123249 together with the strain Lactococcus lactis 1002 (Valio Oy) (composition B), and the strain Lactococcus lactis ssp. lactis biovar. diacetylactis DL2126 VTT E-123249 together with the commercial starter Probat 505 FRO 500 DCU (Danisco) (composition C) were produced by culturing the strain(s)/starter in 500 ml recomposed milk medium supplemented with 0.25% casein hydrolysate, and 1.1% citrate (1.1% trisodium citrate) in the temperature of 30° C. and with 400 rpm mixing for 21 hours. Further 0.025 ml structol was added for preventing foaming. The aeration was 150 ml/min.

Diacetyl contents of the produced milk aroma compositions were about 180 mg/kg (composition A), about 140 mg/kg (composition B), and about 230 mg/kg (composition C) depending on the diacetyl producing strain(s) selection.

It will be obvious to a person skilled in the art that, as the technology advances, the inventive concept can be implemented in various ways. The invention and its embodiments are not limited to the examples described above but may vary within the scope of the claims.

1. A method of preparing an aroma milk composition comprising diacetyl producing strain Lactococcus lactis ssp. lactis biovar. diacetylactis strain DL2126, VTT E-123249, and diacetyl in an amount of at least 50 mg/kg in a milk based medium, the method comprising:
providing a mixture comprising diacetyl producing strain Lactococcus lactis ssp. lactis biovar. diacetylactis strain DL2126, VTT E-123249 and a growth medium comprising dairy; and
incubating the mixture thus obtained for 10 to 28 hours to provide the aroma milk composition comprising diacetyl producing strain Lactococcus lactis ssp. lactis biovar. diacetylactis strain DL2126, VTT E-123249 and diacetyl in an amount of 50 mg/kg at the minimum based on the weight of the growth medium after incubation.

2. The method according to claim 1, wherein the growth medium comprises lactose, citrate, yeast extract and/or casein hydrolysate.

3. The method according to claim 1, wherein the step of incubating the mixture occurs for 16-28 hours.

4. The method according to claim 1, wherein the step of incubating the mixture occurs for 20-26 hours.

5. The method according to claim 1, wherein the growth medium comprises citrate in a concentration of 1.00-1.10%.

6. The method according to claim 1, wherein the growth medium comprises citrate in a concentration of 0.80-1.40%.

7. The method according to claim 1, wherein the growth medium comprises yeast extract and/or casein hydrolysate in a concentration of 0.25-0.5%.

8. The method according to claim 1, wherein the step of incubating the mixture comprises aerating the mixture in a range of 0.4-0.9 vvm.

9. The method according to claim 1, wherein the step of incubating the mixture comprises aerating the mixture in a range of 0.3-1.1 vvm.

10. The method according to claim 1, wherein the step of incubating the mixture comprises aerating the mixture in a range of of 0.55-0.9 vvm

11. The method according to claim 1, wherein the step of incubating the mixture occurs at a temperature in a range of 29 to 31° C.

12. The method according to claim 1, wherein the step of incubating the mixture occurs at a temperature in a range of 27 to 33° C.

13. The method according to claim 1, wherein the step of incubating the mixture occurs at a pH in a range of 4.8-5.4.

14. The method according to claim 1, wherein the step of incubating the mixture occurs at a pH in a range of 5.0-5.2.

15. The method according to claim 1, wherein the aroma milk composition comprises diacetyl in an amount of at least 100 mg/kg in a milk based medium.

16. The method according to claim 1, wherein the aroma milk composition comprises diacetyl in an amount of at least 150 mg/kg in a milk based medium.

17. The method according to claim 1, wherein the aroma milk composition comprises diacetyl in an amount of at least 200 mg/kg in a milk based medium.

18. The method according to claim 1, wherein the the mixture comprising diacetyl producing strain Lactococcus lactis ssp. lactis biovar. diacetylactis strain DL2126, VTT E-123249 and the growth medium comprises at least two diacetyl producing strains selected from the group consisting of Lactococcus lactis ssp. lactis biovar. diacetylactis strains and L. lactis strains.

19. A dairy product containing the aroma milk composition prepared according to claim 1.

20. The dairy product according to claim 19, wherein the dairy product is a milk fat product, a mixed fat product, a margarine product, an edible fat, a fat blend, butter, a spread, a fresh/soft cheese, a cream, a mayonnaise or sour milk.
